(19) United States
(12) Patent Application Publication
(10) Pub. No.: US 2013/0053882 A1
(43) Pub. Date: Feb. 28, 2013

(54) EMBOLIC MATERIAL EXCISION TRAPPING DEVICE

(71) Applicants: Gordon Donald Hocking, Chiba-ken (JP); Zhenghui Cheng, Guangdong (CN)

(72) Inventors: Gordon Donald Hocking, Chiba-ken (JP); Zhenghui Cheng, Guangdong (CN)

(73) Assignee: ACCESS POINT TECHNOLOGIES, INC., Tokyo, (JP)

(21) Appl. No.: 13/639,315

(22) PCT Filed: Apr. 13, 2010
(86) PCT No.: PCT/JP2010/002673

§ 371 Date: Nov. 2, 2012

(51) Int. Cl. A61F 2/01

(52) U.S. Cl. 606/200

(57) ABSTRACT

To provide an embolic material excision trapping device capable of excising an embolic material in an arteriole or vessel without blocking the blood flow, and further, trapping the material with reliability. A device for removing an embolic material in a lumen in a living body has a long shaft member 1 having a front end and a base end and sending the front end to a distal end of the lumen, a filter member 5 which is provided on the front-end side of the shaft member 1, and contracts linearly inside a catheter for carrying the device while expanding when released into the lumen from the catheter, and a coil member 7 which is provided in the shaft member 1 on the side closer to the base-end side than the filter member 5, and contracts linearly inside the catheter while expanding spirally when released into the lumen from the catheter, where the coil member 7 destroys and traps an embolic material in the lumen, and the filter member 5 traps an liberated piece of the embolic material.

### TECHNICAL FIELD

The present invention relates to an embolic material excision trapping device to excise and trap an embolic material such as a thrombus and the like in a lumen in a living body.

### BACKGROUND ART

Cerebral infarction is a disease occurring when an embolic material (such as a thrombus, fatty embolic material, tumor embolic material and the like) enters an artery of a brain, narrows the artery and blocks the blood flow, and cerebral ischemia arises. When the blood flow to the brain is blocked by the embolic material, supplies of oxygen and nutrition to brain cells are interrupted, and the brain cells become necrotic in a short time. Therefore, it is important to secure the normal blood flow promptly in the early stage of the onset of cerebral infarction. Unless the normal blood flow is secured immediately, brain tissue will die, the function of the dead portion is lost, and the life threatening risk to the patient becomes high.

As treatment for cerebral infarction, generally, in an early stage within the first three to six hours after the onset, used is a cerebral embolic material dissolving remedy for injecting a drug directly into the vein, artery or obturator artery to dissolve or transfer the embolic material so as to open again the blood vessel. However, the cerebral embolic material dissolving remedy or medication has a possibility that the embolic material cannot be dissolved completely. Further, such a case may occur that the transferred embolic material flows into the peripheral side of the lesion to block the blood flow again, and that a severe complication thereby occurs.

Then, in recent years, the advance of intravascular treatment has contributed to implementation of methods for surgically securing the blood flow or removing the embolic material. For example, the techniques used are Percutaneous Transluminal Angioplasty for dilating a blood vessel using a balloon microcatheter, stenting for covering a balloon with a mesh-shaped tube called a stent, inflating the balloon to fix the stent to the blood vessel, indwelling the stent after removing the balloon, and supporting the wall of the blood vessel from the inside to prevent the narrowing, and the like.

Further, a device has been developed where wire is directly drawn into a narrowed location of the blood vessel and the embolic material is trapped by a basket, filter or the like provided in the wire (for example, see Patent Document 1).

### CITATION LIST

### Patent Literature

[PTL 1]Japanese Laid-Open Patent Publication No. 2004-097807

### SUMMARY OF INVENTION

### Technical Problem

However, in the conventional Percutaneous Transluminal Angioplasty and stenting as described above, even when the blood flow is secured by widening the blood vessel, there is the risk that the embolic material that is liberated during the operation blocks again the blood flow on the peripheral side of the lesion. Further, these methods require temporarily blocking the blood flow using a balloon or the like to perform the treatment, and have the problem of increasing the adverse effect on the patient as the time elapses.

Meanwhile, the conventional method for trapping an embolic material using a basket or filter is capable of being implemented without blocking the blood flow, but does not have the function of actively excising an embolic material.

In view of the above-mentioned conventional problems, it is an object of the invention to provide an embolic material excision trapping device capable of excising an embolic material in an arteriole or vessel without blocking the blood flow, and further, trapping the material with reliability.

### Solution of Problem

In order to solve the above-mentioned problems, the invention provides an embolic material excision trapping device which is a device to remove an embolic material in a lumen in a living body, and which is characterized by having a long shaft member having a front distal end and a base proximal end and sending the front distal end to a distal end of the lumen, a filter member which is provided on the front-end side of the shaft member, and contracts linearly inside a catheter for carrying the device while self expanding when released into the lumen from the catheter, and a coil member which is provided in the shaft member on the side closer to the proximal-end side than the filter member, and contracts linearly inside the catheter while expanding spirally when released into the lumen from the catheter, where the coil member ensnares and traps embolic material in the lumen, and the filter member traps any liberated or dislodged fragments of the embolic material.

Then, a binding portion on the front-end side of the filter member is fixed to the shaft member, and another binding portion on the base-end side is provided slidably along the shaft member. Alternately, a binding portion on the front-end side and another binding portion on the base-end side of the filter member may be provided slidably along the shaft member, while a stopper member fixed to the shaft member is provided between the binding portion on the front-end side and the binding portion on the base-end side.

Herein, the filter member is shaped in a substantially elliptical form made from mesh woven or braided of metal wires having elasticity or shape memory properties, and has binding portions for binding the wires to the shaft member respectively on the front-end side and the base-end side, and in expanding, it is preferable that the binding portion on the front-end side and the binding portion on the base-end side approach each other along the shaft member, and that the substantially elliptical form is folded in a concave manner toward the base-end side.

Further, the filter member is comprised of a substantially semi-elliptical filter body made from mesh woven or braided of metal wires having elasticity or shape memory properties, in a concave manner toward the base-end side of the shaft member, and a support wire to support the filter body on the base-end side to the shaft member, and preferably has binding portions for binding the filter body and the support wire to the shaft member on the front-end side and the base-end side.

The coil member is preferably comprised of one to five metal wires having elasticity or shape memory properties. Then, the embolic material excision trapping device is preferably subjected to a heparin coating or a hydrophilic coating.

### Advantageous Effect of Invention

According to the embolic material excision trapping device of the invention, the treatment can be performed while securing the blood flow without blocking the blood flow with a balloon or the like, and it is thereby possible to minimize the adverse effect on a patient.

Further, since the excising coil and filter are arranged in a two-step configuration, it is possible to efficiently remove an embolic material. Particularly, by the filter located behind distal to the excising or capture coil, it is possible to trap also liberated pieces of the embolic material escaping from the excising coil with reliability.

### BRIEF DESCRIPTION OF DRAWINGS

[FIG. 1]

FIG. 1 is an entire schematic view of an embolic material excision trapping device according to Embodiment 1 of the invention.

[FIG. 2]

FIG. 2 is a schematic view showing an overview in using the embolic material excision trapping device according to Embodiment 1 of the invention in a cerebral artery.

[FIG. 3]

FIG. 3 is a (first) schematic view showing an aspect in using the embolic material excision trapping device according to Embodiment 1 of the invention.

[FIG. 4]

FIG. 4 is a (second) schematic view showing an aspect in using the embolic material excision trapping device according to Embodiment 1 of the invention.

[FIG. 5]

FIG. 5 is a schematic view showing a modification of a basket filter in Embodiment 1 of the invention.

[FIG. 6]

FIG. 6 is an entire schematic view of an embolic material excision trapping device according to Embodiment 2 of the invention.

[FIG. 7]

FIG. 7 is a schematic view showing an aspect in using the embolic material excision trapping device according to Embodiment 2 of the invention.

[FIG. 8]

FIG. 8 is an entire schematic view of an embolic material excision trapping device according to Embodiment 3 of the invention.

[FIG. 9]

FIG. 9 is a schematic view showing an aspect in using the embolic material excision trapping device according to Embodiment 1 of the invention.

### DESCRIPTION OF EMBODIMENTS

Embodiments of the invention will specifically be described below with reference to accompanying drawings. In addition, in the description, in a lumen of a living body, an upstream side (side nearer the heart in the case of a blood vessel) is referred to “proximal”, while the downstream side is referred to “distal”.

### EXAMPLE 1

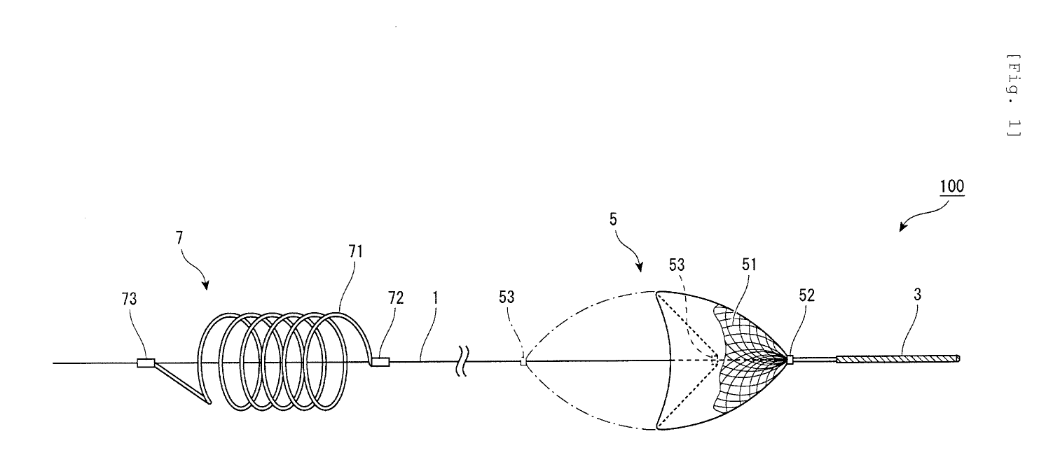

FIG. 1 is an entire schematic view of an embolic material excision trapping device according to Embodiment 1 of the invention. As shown in the figure, the embolic material excision trapping device 100 is formed of a shaft 1, and a guide portion 3, a basket filter 5, and an excising coil 7 provided in the shaft 1. The guide portion 3 is first provided at the front end (distal end) of the shaft 1, and then, the basket filter 5 and excising coil 7 are disposed in this order in the axis direction toward the base end (proximal end) of the shaft 1.

The shaft 1 is formed of a long wire (screw wire), and inserted into a catheter described later. The shaft 1 is movable in the axis direction inside the catheter by operating the base end. Suitable as a material of the shaft 1 is metal such as nickel-titanium alloys, stainless steel, titanium and the like.

At the front end (distal end) of the shaft 1 is provided the guide portion 3 having flexibility. The guide portion 3 is formed of a wire wound in the shape of a coil or woven wires being soft and flexible to guide traveling of the embolic material excision trapping device 100 inside the blood vessel or catheter.

The basket filter 5 is provided on the proximal-end side of the guide portion 3. The basket filter 5 is made from mesh woven of a plurality of metal wires 51 having elasticity or shape memory properties, and as indicated by alternate long and short dashed lines in FIG. 1, is shaped in a substantially elliptical form. Then, by the elasticity or shape memory properties, in a free state, as indicated by solid line and dotted lines in FIG. 1, the basket filter 5 is transformed into a parasol type such that the elliptical form is folded substantially in the center. The mesh made of metal wires 51 is capable of reliably trapping an embolic material while passing the blood flow.

Opposite ends of the plurality of metal wires 51 are bound respectively by binding portions 52 and 53 in the shape of a ring. The binding portion 52 located on the front-end side of the shaft 1 is fixed to the shaft 1, while the binding portion 53 located on the base-end side of the shaft 1 is provided slidably in the axis direction of the shaft 1. Therefore, the basket filter 5 is capable of contracting thinly in a linear manner along the shaft 1 by the binding portion 53 on the base-end side shifting to the base-end side (proximal end). Further, as the binding portion 53 on the base-end side shifts to the binding portion 52 side on the front-end side, the wires 51 forming the basket filter 5 are folded substantially in the center, expand in the radius direction, and are transformed into a parasol type. When transformed into the parasol type, the binding portion 53 on the base-end side is located closer to the binding portion 52 on the front-end side than the folded portion. By thus being folded, the basket filter 5 becomes a double filter, and is capable of trapping an embolic material with higher reliability. In addition, the diameter is preferably in the range of about 2.5 to 3 mm when the filter is transformed into the parasol type.

The wires 51 forming the basket filter 5 are made of nickel-titanium wires, titanium wires, or similar material wires of a composite material of wires made of platinum or gold and nickel-titanium alloy, gold-plated nickel wires, wires of titanium alloy or the like, subjected to heat treatment after being woven or braided, and thus provided with thermosetting properties to be used. The diameter of the wire 51 desirably ranges from about 0.02 to 0.2 mm.

The excising coil 7 is located on the proximal-end side of the basket filter 5. The excising coil 7 is formed of a wire 71 having elasticity or shape memory properties. Then, in a free state, the excising coil 7 maintains a spiral shape as shown in FIG. 1 by the elasticity or shape memory properties. The number of spires of the excising coil 7 is preferably in the range of 2 to 5. Opposite ends of the wire 71 are coupled to the shaft 1 by coupling portions 72 and 73. The coupling portion 72 located on the front-end side of the shaft 1 or the coupling portion 73 located on the rear-end side is slidable in the axis direction of the shaft 1. Meanwhile, the coupling portion 73 located on the base-end side of the shaft 1 is fixed to the shaft 1. Therefore, the excising coil 7 is capable of expanding linearly along the shaft 1 by the coupling portion 72 on the front-end side shifting to the front-end side or rear-end side of the embolic material excision trapping device 100. Further, as another Embodiment, both of the coupling portions 72 and 73 may travel slidably around a fix portion (not shown in the figure) fixed to the shaft 1 inside the basket filter 5.

The wire 71 forming the excising coil 7 is made of nickel-titanium wire, titanium wire, or similar wire of a composite material of wire made of platinum or gold and nickel-titanium alloy, gold-plated nickel wire, wire of titanium alloy or the like. The wire 71 may be subjected to heat treatment to have thermosetting. The diameter of the wire 71 desirably ranges from about 0.02 to 0.1 mm.

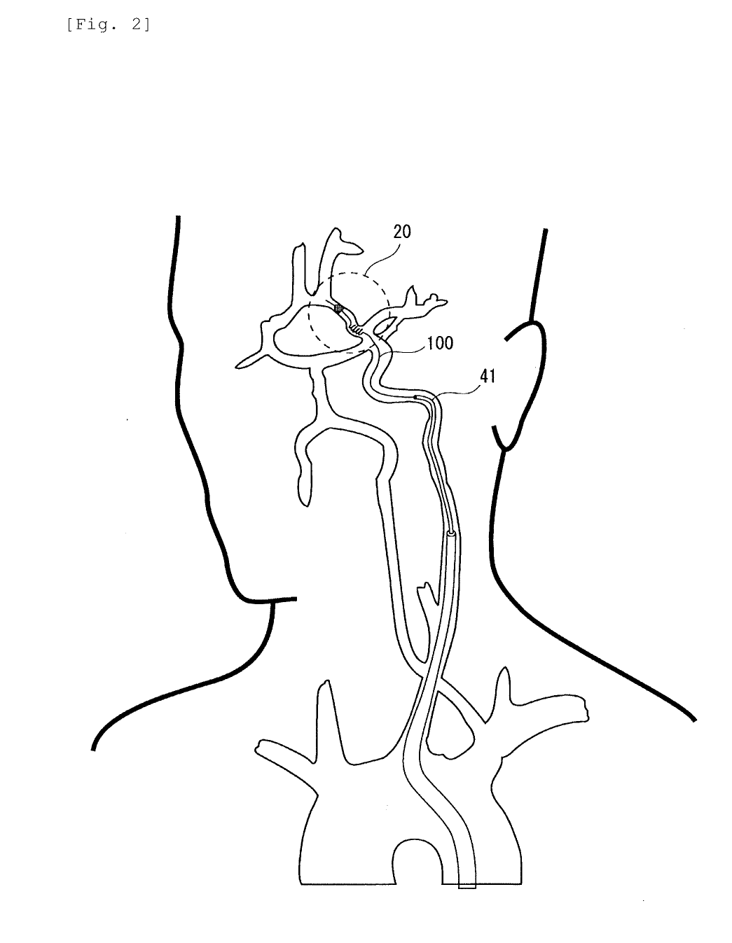

An aspect in using the embolic material excision trapping device of this Embodiment as described above will be described below with reference to FIGS. 2 to 4.

First, as a preliminary step to use the device, a guide wire is introduced into a blood vessel 21 of a lesion 20. Then, a microcatheter 41 is inserted into the blood vessel 21 of the lesion 20 through the guide wire, and the guide wire is removed.

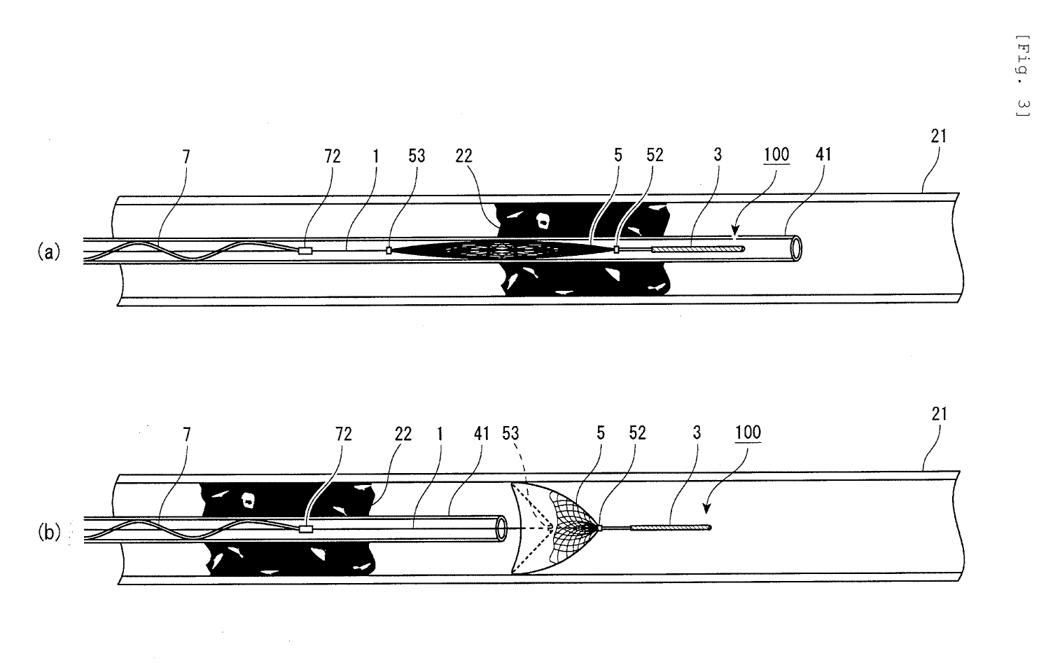

Next, as shown in FIG. 3(a), the embolic material excision trapping device 100 is passed through the microcatheter 41 inserted into the blood vessel 21 of the lesion 20, and transferred to the peripheral side of the lesion (narrowed portion). At this point, inside the microcatheter 41, the binding portion 53 on the base-end side of the basket filter 5 has slid to the base-end side of the shaft 1, and the basket filter 5 has contracted linearly. Similarly, the coupling portion 72 on the front-end side of the excising coil 7 has slid to the front-end side of the shaft 1, and the excising coil 7 has expanded also linearly. Therefore, the embolic material excision trapping device 100 is capable of traveling smoothly inside the microcatheter 41.

Then, as shown in FIG. 3(b), the embolic material excision trapping device 100 is made project from the front end of the microcatheter 41 on the peripheral side of the lesion (narrowed portion). When the basket filter 5 separates from the microcatheter 41, the basket filter 5 automatically expands by its elasticity or shape memory properties, is transformed into a parasol type, and brought into intimate contact with the wall of the blood vessel optimal wall contact or wall opposition.

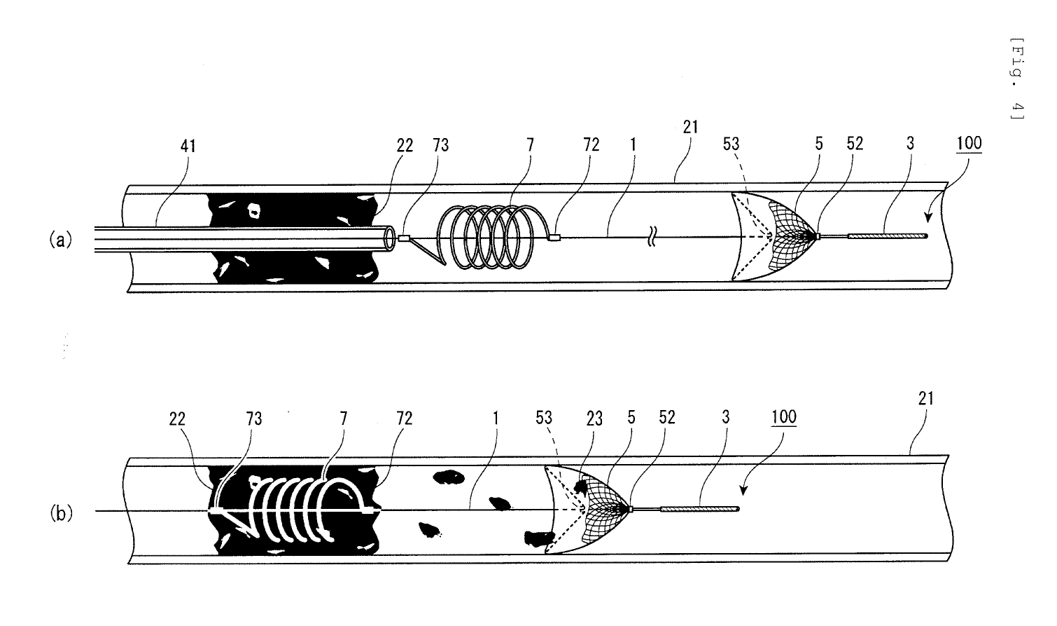

Next, as shown in FIG. 4(a), after the excising coil 7 is removed from the front end of the microcatheter 41, the microcatheter 41 is moved back to the base-end side. When the excising coil 7 separates from the microcatheter 41, the excising coil 7 automatically contracts by its elasticity or shape memory properties, and is restored to an original coil shape as shown in the figure.

Then, as shown in FIG. 4(b), the embolic material excision trapping device 100 is slowly shifted to the base-end direction, and the excising coil 7 destroys and excises the embolic material 22 and then, traps the material. Librated pieces 23 of the embolic material escaped in the excision of the excising coil 7 are trapped by the basket filter 5 located at the back of the excising coil 7.

After finishing the treatment, the microcatheter 41 is pushed with the shaft 1 fixed, the embolic material excision trapping device 100 is stored again in the microcatheter 41, and the microcatheter 41 is withdrawn.

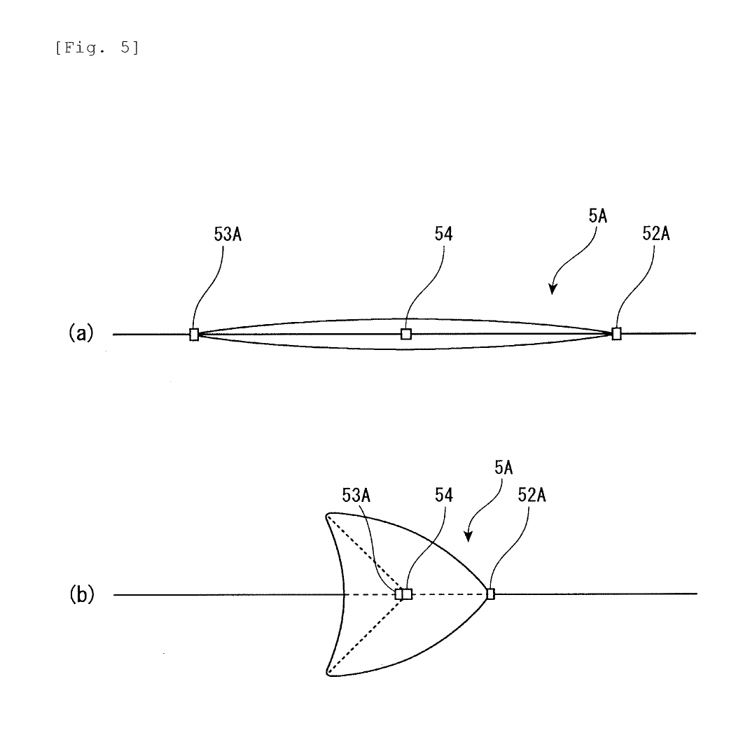

In addition, it is described above that the binding portion 52 on the front-end side of the basket filter 5 is fixed to the shaft 1 while the binding portion 53 on the base-end side is made slidable with respect to the shaft 1, but substituting therefor, both of the binding portions may be made slidable. FIG. 5 shows a structure of a modification of the basket filter. Binding portions 52A and 53A of a basket filter 5A are provided slidably with respect to the shaft 1. A stopper member 54 is fixed to the shaft 1 substantially in the center of the basket filter 5A. By thus configuring, when the basket filter 5A expands, as shown in FIG. 5(b), the binding portion 53A on the base-end side is caught in the stopper member 54, and the basket filter 5A is kept at a predetermined position.

Alternately, both of the binding portions 52 and 53 on the front-end side and base-end side may be fixed to the shaft 1 to eliminate the shaft 1 between the binding portions 52 and 53. The essential structure is only that the basket filter 5 is held at positions in a predetermined range while being able to expand and contract freely.

Similarly, with respect to the excising coil 7, such a structure is described above that the coupling portion 72 on the front-end side is slidable with respect to the shaft 1 while the coupling portion 73 on the base-end side is fixed to the shaft 1, but for example, both of the coupling portions 72 and 73 may be fixed to the shaft 1 to eliminate the shaft 1 between the coupling portions 72 and 73.

In addition, in the above-mentioned embolic material excision trapping device 100, its surface may be coated with a medical agent. For example, for the purpose of preventing blood coagulation, a coating of an anticoagulant (for example, heparin) may be applied. Further, when a hydrophilic coating is applied, the device exhibits lubricity when contacting the blood. The operation for inserting the device into the blood vessel is thereby made easy.

### EXAMPLE 2

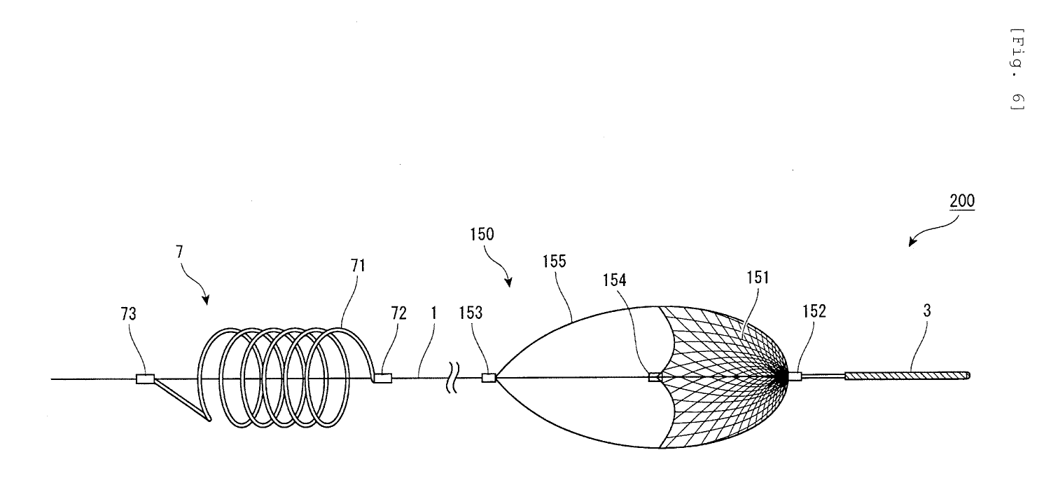

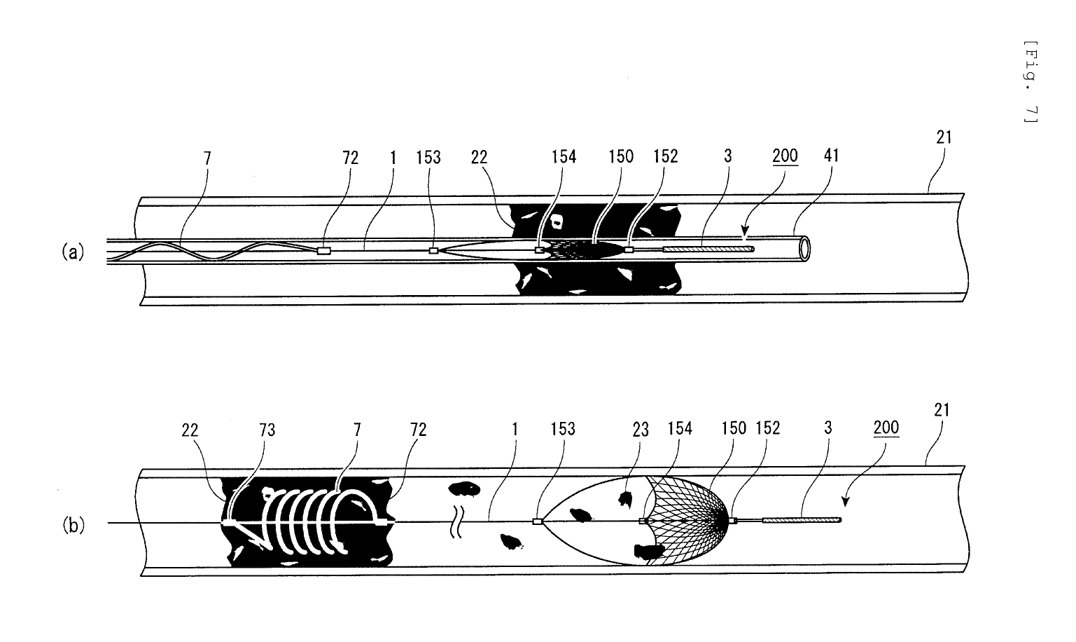

This Embodiment describes an example of using a parachute type basket filter substituting for the parasol type basket filter of Embodiment 1. FIG. 6 is an entire schematic view of an embolic material excision trapping device according to Embodiment 2 of the invention. Further, FIG. 7 is a schematic view showing an aspect in using the embolic material excision trapping device. In addition, the same parts as in Embodiment 1 are assigned the same reference numerals to omit specific descriptions thereof.

In the embolic material excision trapping device 200 of this Embodiment, a basket filter 150 is formed of mesh woven of a plurality of metal wires 151 having elasticity or shape memory properties. A shape of the filter 150 is a substantially elliptical form as shown in FIG. 7, the mesh portion occupies almost a half in the ellipse, and thus, the filter has a parachute-like shape such that support wires 155 support the semi-elliptical mesh portion. The mesh portion corresponding to a parasol of the parachute is capable of trapping librated pieces 23 and the like of the embolic material while securing the blood flow.

The support wires 155 on the proximal-end side are bound by a ring-shaped binding portion (binding portion on the base-end side) 153. Meanwhile, the support wires 155 on the distal-end side are bound together with the wires 155 forming the mesh by a ring-shaped binding portion (binding portion on the front-end side) 152. Each of these binding portions 152 and 153 is provided slidably in the axis direction of the shaft 1. Meanwhile, a stopper member 154 fixed to the shaft 1 is disposed between the binding portions 152 and 153. Therefore, the basket filter 150 is capable of traveling along the shaft 1 in the range limited by the stopper member 154.

Accordingly, the basket filter 150 is capable of contracting thinly in a linear manner along the shaft 1 as shown in FIG. 7(a) when inserted into the microcatheter 41. Meanwhile, when the filter 150 is removed from the microcatheter 41, as shown in FIG. 7(b), the filter 150 automatically expands in the radius direction by its elasticity or shape memory properties, is transformed into the parachute type, and brought into intimate contact with the wall of the blood vessel. The diameter desirably ranges from about 2.5 to 3 mm when the filter is transformed into the parachute type.

The wires 151 forming the mesh of the basket filter 150 are made of nickel-titanium wires, titanium wires, wires of a composite material of wires made of platinum or gold and nickel-titanium alloy, gold-plated nickel wires, wires of titanium alloy or the like, subjected to heat treatment after being woven or braided, and thus provided with thermosetting to be used. The diameter of the wire 151 desirably ranges from about 0.02 to 0.2 mm.

In addition, such a structure is described above that the binding portions 152 and 153 of the basket filter 150 are made slidable with respect to the shaft 1 while the stopper member 154 is provided between the binding portions 152 and 153, but another structure may be applied that one of the binding portions 152 and 153 is fixed while the other one is made slidable without using the stopper member 154. Alternately, both of the binding portions 152 and 153 on the front-end side and base-end side may be fixed to the shaft 1 to eliminate the shaft 1 between the binding portions 152 and 153. The essential structure is only that the basket filter 150 is held at positions in a predetermined range while being able to expand and contract freely.

As in Embodiment 1, in the above-mentioned embolic material excision trapping device 200, its surface may be coated with a medical agent. For example, for the purpose of preventing blood coagulation, a coating of an anticoagulant (for example, heparin) may be applied. Further, when a hydrophilic coating is applied, the device exhibits lubricity when contacting the blood. The operation for inserting the device into the blood vessel is thereby made easy.

### EXAMPLE 3

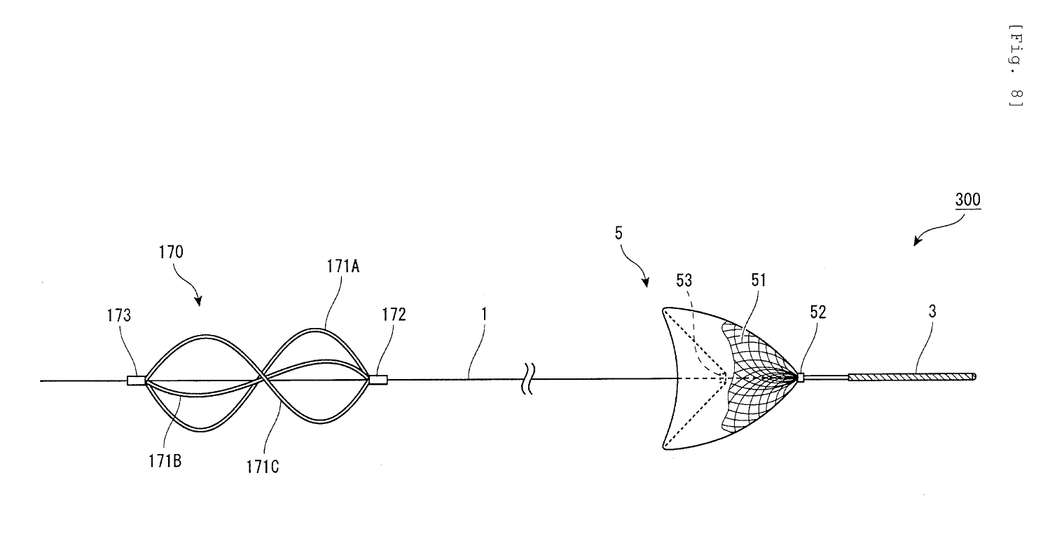

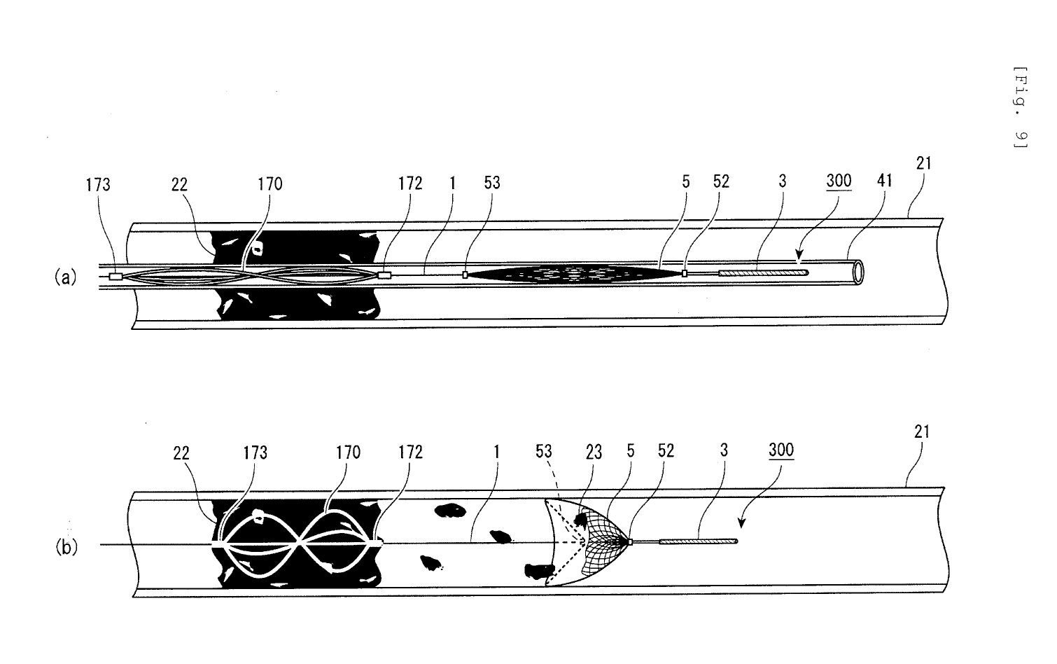

This Embodiment describes an example of using an excising coil formed of a plurality of wires substituting for the excising coil formed of a single wire of Embodiment 1. FIG. 8 is an entire schematic view of an embolic material excision trapping device according to Embodiment 3 of the invention. Further, FIG. 9 is a schematic view showing an aspect in using the embolic material excision trapping device. In addition, the same parts as in Embodiment 1 are assigned the same reference numerals to omit specific descriptions thereof.

In the embolic material excision trapping device 300 of this Embodiment, an excising coil 170 is formed of a plurality of wires 171 having elasticity or shape memory properties. The wires 171 maintain a spiral shape as shown in FIG. 8 by the elasticity or shape memory properties in a free state. It is preferable to use three to five wires as the plurality of wires 171 (shown herein is the example of using three wires, 171A, 171B and 171C.) The diameter of a spire of the wire 171 preferably ranges from about 2.5 to 3 mm. Opposite ends of each wire 171 are coupled to the shaft 1 by coupling portions 172 and 173. The coupling portion 172 located on the front-end side of the shaft 1 is slidable in the axis direction of the shaft 1. Meanwhile, the coupling portion 173 located on the base-end side of the shaft 1 is fixed to the shaft 1. Therefore, the excising coil 170 is capable of expanding linearly along the shaft 1 by the coupling portion 172 on the front-end side shifting to the front-end side of the shaft 1. Accordingly, the excising coil 170 expands thinly in a linear manner along the shaft 1 as shown in FIG. 9(a) when introduced to inside the microcatheter 41. Meanwhile, when the coil 170 is discharged from the microcatheter 41, as shown in FIG. 9(b), the coil automatically expands by its elasticity or shape memory properties, and is transformed into the spiral shape. Then, the expanded excising coil 170 destroys and excises the embolic material 22, and then, traps the material 22.

In addition, such a structure is described above that the coupling portion 172 on the front-end side of the excising coil 170 is made slidable with respect to the shaft 1 while the coupling portion 173 on the base-end side is fixed to the shaft 1, but both of the binding portions 172 and 173 on the front-end side and base-end side may be fixed to the shaft 1 to eliminate the shaft 1 between the coupling portions 172 and 173. The essential structure is only that the excising coil 170 is limited in position while being able to expand and contract freely.

The wires 171 forming the excising coil 170 are made of nickel-titanium wires, titanium wires, wires of a composite material of wires made of platinum or gold and nickel-titanium alloy, gold-plated nickel wires, wires of titanium alloy or the like. The wires 171 may be subjected to heat treatment to have thermosetting. The diameter of the wire 171 desirably ranges from about 0.02 to 0.1 mm.

In the above-mentioned embolic material excision trapping device 300, as in Embodiments 1 and 2, its surface may be coated with a medical agent. For example, for the purpose of preventing blood coagulation, a coating of an anticoagulant (for example, heparin) may be applied. Further, when a hydrophilic coating is applied, the device exhibits lubricity when contacting the blood. The operation for inserting the device into the blood vessel is thereby made easy.

In addition, the subject described in the forgoing is mainly excision and trapping of embolic materials in cerebral arteries and carotid arteries in treatment for cerebral infarction, but the invention is not limited thereto in application thereof, and for example, is applicable to excision and trapping of an embolic material in a coronary artery in cardiac infarction, and removal of embolic materials in various lumens in a body such as extraction of gallstones in a bile duct and the like.

Since the device is configured as described above, according to the embolic material excision trapping apparatus of the invention, the treatment is capable of being performed with the blood flow secured without blocking the blood flow using a balloon or the like, and it is thereby possible to minimize the adverse effect on a patient.

Further, since the excising coil and filter are arranged in a two-step configuration, it is possible to efficiently remove an embolic material. Particularly, by the filter located at the back of the excising coil, it is possible to trap also liberated pieces of the embolic material escaping from the excising coil with reliability.

The Embodiments of the present invention are described above, but the invention is not limited to the above-mentioned Embodiments, and various modifications are capable of being carried out based on the subject matter of the invention, and are not excluded from the scope of the invention.

### INDUSTRIAL APPLICABILITY

### Industrial Applicability

The present invention relates to an embolic material excision trapping device to excise and trap an embolic material such as a thrombus and the like in a lumen in a living body, and has the industrial applicability.

### REFERENCES SIGNS LIST

1 Shaft

3 Guide portion

5, 5A, 150 Basket filter

7, 170 Excising capture coil

20 Lesion

21 Blood vessel

22 Embolic material

23 Librated piece

41 Microcatheter

51, 151 Wire

52, 52A, 152 Binding portion on the front-end side

53, 53A, 153 Binding portion on the base-end side

54, 154 Stopper member

71, 171 Wire

72, 172 Coupling portion on the front-end side

73, 173 Coupling portion on the base-end side

100, 200, 300 Embolic material excision trapping device

155 Support wire

1. An embolic material excision trapping device to remove an embolic material in a lumen in a living body, comprising:
a long shaft member having a front end and a base end and sending the front end to a distal end of the lumen;
a filter member which is provided on the front-end side of the shaft member, and contracts linearly inside a catheter for carrying the device while expanding when released into the lumen from the catheter; and
a coil member which is provided in the shaft member on the side closer to the base-end side than the filter member, and contracts linearly inside the catheter while expanding spirally when released into the lumen from the catheter,
wherein the coil member destroys and traps embolic material in the lumen, and the filter member traps a liberated of the embolic material.

2. The embolic material excision trapping device according to claim 1, wherein a binding portion on the front-end side and another binding portion on the base-end side of the filter member are provided slidably along the shaft member, and a stopper member fixed to the shaft member is provided between the binding portion on the front-end side and the binding portion on the base-end side.

3. The embolic material excision trapping device according to claim 1, wherein the filter member is shaped in a substantially elliptical form made from mesh woven of metal wires having elasticity or shape memory property, and has binding portions for binding the wires to the shaft member respectively on the front-end side and the base-end side, and
when the filter member expands, the binding portion on the front-end side and the binding portion on the base-end side approach each other along the shaft member, and the substantially elliptical form is folded in a concave manner toward the base-end side.

4. The embolic material excision trapping device according to claim 1, wherein the filter member is comprised of a substantially semi-elliptical filter body made from mesh woven of metal wires having elasticity or shape memory property, in a concave manner toward the base-end side of the shaft member, and a support wire to support the filter body on the base-end side to the shaft member, and
has binding portions for binding the filter body and the support wire to the shaft member on the front-end side and the base-end side.

5. The embolic material excision trapping device according to claim 1, wherein the binding portion on the front-end side of the filter member is fixed to the shaft member, and the binding portion on the base-end side is provided slidably along the shaft member.

6. The embolic material excision trapping device according to claim 1, wherein the coil member is comprised of one to five metal wires having elasticity or shape memory property.

7. The embolic material excision trapping device according to claim 1, wherein the embolic material excision trapping device is subjected to a heparin coating or a hydrophilic coating.
